(19) United States
(12) Patent Application Publication
(10) Pub. No.: US 2019/0307107 A1
(43) Pub. Date: Oct. 10, 2019

(54) COMPOSITIONS AND METHODS FOR MODELING HEART FAILURE WITH PRESERVED EJECTION FRACTION

(71) Applicant: THE BOARD OF REGENTS OF THE UNIVERSITY OF TEXAS SYSTEM, Austin, TX (US)

(72) Inventors: Joseph A HILL, Dallas, TX (US); Gabriele G SCHIATTARELLA, Dallas, TX (US); Thomas G GILLETTE, Dallas, TX (US)

(73) Assignee: THE BOARD OF REGENTS OF THE UNIVERSITY OF TEXAS SYSTEM, Austin, TX (US)

(21) Appl. No.: 16/309,026

(22) PCT Filed: Jun. 12, 2017
(86) PCT No.: PCT/US17/37019

§ 371 Date: Dec. 11, 2018

Related U.S. Application Data
(60) Provisional application No. 62/349,319, filed on Jun. 13, 2016.

(51) Int. Cl. A01K 67/02; A61N 1/362; A23K 20/147; A23K 20/158; A23K 20/163; A23K 20/174; A23K 50/50; A61K 31/575

(52) U.S. Cl. CPC A01K 67/02; A61N 1/362; A23K 20/147; A23K 20/158; A61K 45/06; A23K 20/174; A23K 50/50; A61K 31/575; A23K 20/163

(57) ABSTRACT

Aspects of the disclosure relate to a composition comprising 20-60% w/w of fat combined with a nitric oxide synthase inhibitor. Method aspects of the disclosure relate to a method for inducing heart failure with preserved ejection fraction in an experimental laboratory animal, the method comprising administering a composition of the disclosure or a composition comprising 10-60% w/w of fat and a composition comprising a nitric oxide synthase inhibitor to the laboratory animal.

This application claims priority to U.S. Provisional Patent Application Ser. No. 62/349,319, filed Jun. 13, 2016, which is incorporated by reference herein in its entirety.

### BACKGROUND OF THE INVENTION

### I. Field of the Invention

Embodiments are directed generally to biology and veterinary food compositions. In certain aspects there are methods and compositions for modeling heart failure with preserved ejection fraction.

### II. Background

Heart failure (HF) is the most prevalent form of cardiac disease in the US. It is defined as the condition in which the heart cannot pump enough blood to meet the body's needs. This can happen in two forms: a) when the heart does not contract effectively, called HF with reduced ejection fraction (HFrEF); and b) when the heart contractility is normal but filling is perturbated, called HF with preserved ejection fraction (HFpEF).

In the US, 40% of patients with heart failure have HFpEF. These patients commonly present with obesity, diabetes, hypertension, exercise intolerance and pulmonary congestion. In addition, multiple lines of evidence suggest that nitric oxide (NO) dysregulation plays an important role in HFpEF pathophysiology. So far, there is not a single effective treatment targeting this population. While therapies like beta-blockers and angiotensin converting enzyme (ACE) inhibitors are standard of care for HFrEF, these treatments do not benefit the 40% of HF patients with HFpEF.

The main reason why such an important need has not been met is the lack of relevant experimental models to study HFpEF biology and test potential therapies. Because of the need to measure cardiac function, it is virtually impossible to study HFpEF in a culture dish. Additionally, HFpEF is considered a “whole-body condition” in nature, which supports the usage of pre-clinical animal studies. Therefore, there is a need in the art for methods that effectively model this disease in mammalian species in vivo.

### SUMMARY OF THE INVENTION

The current disclosure fulfills the aforementioned need in the art by providing compositions and methods that can recapitulate HFpEF in laboratory animals. Accordingly, aspects of the disclosure relate to a composition comprising 20-60% w/w of fat and a nitric oxide synthase inhibitor.

In some embodiments, the w/w % of fat is at least, at most, or exactly 10, 20, 30, 40, 50, 60, or 75% w/w, or any derivable range therein.

In some embodiments, the nitric oxide synthase inhibitor is N^{ω}-Nitro-L-arginine methyl ester (L-NAME) or a salt thereof. In some embodiments, the composition comprises about 0.05-0.10% w/w of nitric oxide synthase inhibitor. In some embodiments, the composition comprises about 0.01-0.50% w/w of L-NAME. In some embodiments, the composition comprises 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.35, 0.4, 0.45, or 0.5% w/w of L-NAME (or any derivable range therein) or of a nitric oxide synthase inhibitor. In some embodiments, the composition comprises 27-37% w/w of fat combined with 0.05-0.1% w/w L-NAME or a salt thereof.

In some embodiments, the composition comprises at least 0.01% w/w cholesterol. In some embodiments, the composition comprises at least, at most, or exactly 0.005, 0.01, 0.02, 0.03, 0.04, 0.05, 0.075, 0.1, 0.2, 0.3, or 0.4% w/w of cholesterol (or any range derivable therein). In some embodiments, the composition comprises 0.01-0.05% w/w cholesterol.

In some embodiments, the composition further comprises 10-30% w/w protein. In some embodiments, the composition comprises at least, at most, or exactly 5, 10, 15, 20, 25, 30, 35, 40, 45, 50, or 60% w/w, or any derivable range therein, of protein.

In some embodiments, the composition further comprises 10-30% w/w carbohydrate. In some embodiments, the composition comprises at least, at most, or exactly 5, 10, 15, 20, 25, 30, 35, 40, 45, 50, or 60% w/w, or any derivable range therein, of carbohydrate.

In some embodiments, the composition further comprises minerals and/or vitamins. In some embodiments, the compositions comprise at least, at most, or exactly 1, 2, 3, 4, 5, 6, 7, 8, 9, 10, 12, 14, 16, 18, or 20% w/w of vitamins and/or minerals (or any derivable range therein).

In some embodiments, the composition is formulated for oral ingestion. In further embodiments, the composition is formulated for subcutaneous, intramuscular, intradermal, intraepidermal, intravenous or intraperitoneal administration. In some embodiments, the composition is formulated for animal consumption.

In some embodiments, the composition comprises: 0.05-0.1% w/w of L-NAME or a salt thereof; 27-37% w/w of fat; 10-30% w/w protein; and 10-30% w/w carbohydrate. In some embodiments, the protein comprises an ingredient described herein. In some embodiments, the amino acid is L-Cystine. In some embodiments, the protein comprises one or both of Casein and amino acids. In some embodiments, the composition further comprises a starch. In some embodiments, the composition comprises one or more of corn starch, maltodextrin, sucrose, and cellulose. In some embodiments, the starch is a starch described herein. In some embodiments, the composition comprises soybean oil and/or lard. In some embodiments, the lard comprises 0.5-1% w/w of cholesterol. In some embodiments, the composition further comprises one or more of di-calcium phosphate, calcium carbonate, potassium citrate, and choline bitartrate.

Method aspects of the disclosure relate to a method for inducing heart failure with preserved ejection fraction in an experimental laboratory animal, the method comprising administering a composition of the disclosure or a composition comprising 10-60% w/w of fat and a composition comprising a nitric oxide synthase inhibitor to the laboratory animal. In some embodiments, the w/w % of fat is at least, at most, or exactly 10, 20, 30, 40, 50, 60, or 75% w/w, or any derivable range therein. In some embodiments, the nitric oxide synthase inhibitor is L-NAME w/w or a salt thereof. In some embodiments, the composition comprises about 0.05-0.1% w/w of nitric oxide synthase inhibitor. In some embodiments, the nitric oxide synthase inhibitor is administered to the animal in a food product. In some embodiments, the nitric oxide synthase inhibitor and the fat are administered in the same composition. In some embodiments, the nitric oxide synthase inhibitor and the fat are administered in the same food product. In some embodiments, the nitric oxide synthase inhibitor is admistered to the animal in a solution. In some embodiments, the nitric oxide synthase inhibitor is administered to the animal in the drinking water. In some embodiments, the dose of L-NAME administered to induce HFpEF is about 120 mg/kg body weight. In some embodiments, the dose of L-NAME is about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 290, 300, 310, 320, 330, 340, or 350 mg/kg (or any derivable range therein). In some embodiments, the dose is a daily dose. In some embodiments, the heart failure is characterized by normal or near normal systolic function. In some embodiments, the heart failure is characterized by obesity and/or insulin resistance. In some embodiments, the heart failure is characterized by hypertension. In some embodiments, the systolic and/or diastolic blood pressure is increased by at least 10 points. In some embodiments, the systolic and/or diastolic blood pressure is increased by at least 10, 12, 14, 16, 18, 20, 22, 24, 26, 28, 30, 32, 34, 36, 38, 40, 42, 44, 46, 48, 50, 52, 54, 56, or 60 points, or any derivable range therein. In some embodiments, the heart failure is characterized by one or more of normal or near normal ejection fraction, diastolic dysfunction, impaired systolic function with global longitudinal strain reduction, exercise intolerance, cardiomyocyte hypertrophy, reduced cardiomyocyte contractility and relaxation, increased cardiac fibrosis, increased vascular stiffness, impaired coronary flow reserve, increased atrial fibrillation episodes and lung congestion.

In some embodiments, the method further comprises determining the expression of a protein or mRNA of a gene in cells and/or tissue of the experimental animal. In some embodiments, the method further comprises determining cardiac metabolism, vascular metabolism, skeletal metabolism, kidney metabolism, liver metabolism, and or microbiome evaluation in the experimental animal. In some embodiments, the method further comprises administering a compound to the laboratory animal. In some embodiments, the method further comprises determining the time course of the absorption of the compound, determining the biological distribution of the compound, determining the metabolism of the compound, and/or determining the excretion on the compound. In some embodiments, the method further comprises performing one or more assays selected from echocardiography, magnetic resonance imaging (MRI), computerized tomography (CT) scan, single-photon emission computed tomography (SPECT)/positron emission tomography (PET) scan, nuclear magnetic resonance (NMR), left and right ventricles catheterization, hemodynamic studies, vascular stiffness measurement, blood glucose measurment, exercise testing, coronary flow reserve measurement, histological evaluation of cardiac morphology, skeletal muscle force measurement on the experimental animal or a biological sample from the experimental animal.

It is contemplated that the compositions of the current disclosure can include any ingredient or any combination thereof described throughout this specification. The concentrations of any ingredient within the compositions can vary. In non-limiting embodiments, for example, the compositions can comprise, consist essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0125%, 0.0150%, 0.0175%, 0.0200%, 0.0275%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims.

Use of the one or more compositions may be employed based on methods described herein. Other embodiments are discussed throughout this application. Any embodiment discussed with respect to one aspect of the disclosure applies to other aspects of the disclosure as well and vice versa. The embodiments in the Example section are understood to be embodiments that are applicable to all aspects of the technology described herein.

The term “effective amount” refers to an amount that achieves a certain effect, such as an increase in systolic or diastolic blood pressure or any effect, particular one related to heart failure pathology, described herein.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

The terms “a,” “and,” and “and/or” in the claims is also used to mean that the claims may comprise any one of the recited elements, all of the recited elements, or any combination thereof.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. HFD+L-NAME (from now on HFpEF) mice exhibit obesity (FIG. 1A) and glucose intolerance (FIG. 1B-C). BW=body weight; g=grams; HFD=high fat diet; IP GTT=intraperitoneal glucose tolerance test; mg/dl=milligrams/deciliter

FIGS. 2A-B. HFpEF mice have increased systolic blood pressure (SBP) (FIG. 2A) and increased diastolic blood pressure (DBP) (FIG. 2B). mmHg=millimeters mercury.

FIG. 3A-B. HFpEF mice have preserved ejection fraction (EF %) (FIG. 3A) with impaired systolic function measured by global longitudinal strain (GLS) (FIG. 3B).

FIG. 4A-C. HFpEF mice have diastolic dysfunction. E/A=ratio of early to late transmitral flow velocities (FIG. 4A-B); E/E′=ratio of early transmitral flow velocity to early diastolic septal mitral annulus velocity (FIG. 4A-C).

FIG. 5A-C. HFpEF mice have exercise intolerance (FIG. 5A); cardiac hypertrophy (FIG. 5B) and signs of heart failure (FIG. 5C). LW=lung weight; HW=heart weight; TL=tibial length; mg/mm=milligrams/millimeter.

FIG. 6A-B. HFpEF mice have cardiomyocytes hypertrophy (FIG. 6A) and increased fibrosis (FIG. 6B) μm²=micrometers squared. WGA=wheat germ agglutinin.

FIG. 7A-C. HFpEF mice have increased vascular stiffness (pulse wave velocity−PWV) (FIG. 7A) and impaired coronary flow reserve (FIG. 7B-C). cm/s=centimeters per second. Iso=isofluorane.

FIG. 8A-B. HFpEF mice are more susceptible to induction of atrial fibrillation (Afib) (FIG. 8A) with an increase of the duration of episodes (FIG. 8B).

FIG. 9A-D. Cardiomyocytes isolated from HFpEF mice have impaired contractility (FIG. 9A-B) and reduced relaxation (FIGS. 9A, C, and D) μm=micrometers; ms=milliseconds.

FIG. 10A-C. COMBO diet induces obesity (FIG. 10A) and glucose intolerance (FIG. 10B-C) in mice. BW=body weight; g=grams; HFD=high fat diet; IP GTT=intraperitoneal glucose tolerance test; mg/dl=milligrams/deciliter.

FIG. 11A-B. COMBO diet increases systolic blood pressure (SBP) (FIG. 11A) and diastolic blood pressure (DBP) (FIG. 11B) in mice. mmHg=millimeters mercury.

FIG. 12A-C. Mice on COMBO diet exhibit preserved ejection fraction (EF %) (FIG. 12A) with impaired diastolic function. E/A=ratio of early to late transmitral flow velocities (FIG. 12B); E/E′=ratio of early transmitral flow velocity to early diastolic septal mitral annulus velocity (FIG. 12C).

FIG. 13A-C. COMBO diet induces exercise intolerance (FIG. 13A); cardiac hypertrophy (FIG. 13B) and signs of heart failure (FIG. 13C). LW=lung weight; HW=heart weight; TL=tibial length; mg/mm=milligrams/millimeter.

### DETAILED DESCRIPTION OF THE INVENTION

The current methods for modeling heart failure with preserved ejection fraction in a laboratory setting are insufficient for effective drug discovery. Available models fail to recapitulate many aspects of this higly complex phenotype and can exhibit renal disease, making them a biased pre-clinical model. The inventors of the application have discovered a composition and method for accurately modeling HF such as HFpEF, and these are described herein.

### I. Feed Particles

The current disclosure includes compositions that may be used for animal consumption, such as animal feed particles. In particular, the compositions are useful for administration to laboratory animals for the induction of heart failure with preserved ejection fraction for the purposes of experimental modeling of the disease. The compositions described herein have a high fat content and comprise a nitric oxide synthase inhibitor such as L-NAME.

L-NAME refers to N^{ω}-Nitro-L-arginine methyl ester and has the structure:

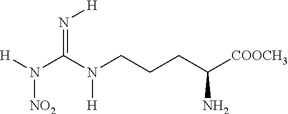 [SMILES: [H]/N=C(\NCCC[C@H](N)C(=O)OC)N[N+](=O)[O-]]

The compositions of the disclosure generally include a high fat content. The fat included in the particles may include more than one fat source. In some embodiments, the compositions are in the form of a feed particle. In some embodiments, a combination of at least two, three, four, or five fats are used. In some embodiments, the feed particles are extruded feed particles.

Feed particles may be made by methods known in the art. For example, feed particles may be made by methods that include mixing the particle ingredients to form a mixture, conditioning the mixture prior to extrusion, extruding feed particles and placing extruded particles into a vacuum coater to incorporate additional fat into the extruded particles. A low melting point fat may be added into a vacuum coater followed by partial release of the vacuum to allow the low melting point fat to enter into the particles. The low melting point is generally in the interior of the particles. A high melting point fat can then be introduced into the vacuum coater and the remaining vacuum released. Some of the high melting point fat can enter the outer region of the particles but, more importantly, the high melting point fat is generally on the exterior and forms a coating on the exterior of the particles that hardens at ambient temperature. The coated extruded particles formed in this manner contain a high amount of fat.

The compositions of the current disclosure can be provided as daily feed ration for a variety of laboratory animals. The laboratory animals can include, for example, mice, rats, donkeys, pigs, dogs, cats, rabbits, horses, sheep, goats, monkeys and non-human primates. In some embodiments, the laboratory animal is a mammal. In embodiments of the disclosure, the term laboratory animal excludes humans.

The compositions of the disclosure may include fat, nutritional components and other additives. Nutritional components can include starch, carbohydrates, and protein components. Other additives can include, for example, amino acids, vitamins, minerals, nutraceuticals, pharmaceuticals and the like. During formation of the particles, the additives may be added into the nutritional components or they may be added to the fat component.

The compositions of the disclosure also include nutritional components. The nutritional components can include starch, carbohydrate, vitamins, minerals, and protein components. Generally, the nutritional components and the additives make up the remaining weight of the particle after taking into account the weight percentage of the fat and L-NAME.

The compositions may comprise starches such as corn, wheat, barley, oats, sorghum, tapioca, isolated dry or wet milled starch, their milled components and combination of any two or more of these. The amount of starch in the particles can vary and is generally at least about 5 percent by weight of the particles. In some preferred embodiments, the amount of starch is at least, at most, or exactly 5, 10, 15, 20, 25, 30, 35, 40, 45, 50, 55, 60, or 65% w/w, or any derivable range therein.

The compositions of the disclosure may also comprise protein components. A wide assortment of protein sources can be included in the compositions and include soybean meal, amino acids, casein, cottonseed meal, and corn gluten meal. Other proteinaceous sources include other oil seed meals such as palm meal; animal by-product meals such as meat meal, poultry meal, blood meal, feather meal and fishmeal; plant by-product meals such as wheat middlings, soybean hulls and corn by-products; and microbial protein such as torula yeast and brewer's yeast. The amount of protein in the compositions can vary.

As described herein, the compositions include particles with a high amount of fat. The fat that is included can be animal fat, vegetable fat, fractionated fat, hydrogenated fat, and/or fats that contain palmitic acid, stearic acid, lauric acid, myristic acid, cocoa butter and any hydrogenated fat or oil. Fats also include cholesterol, linoleic acid, linolenic acid, arachidonic acid, omega-3 fatty acids, saturated fatty acids, and/or monounsaturated fatty acids.

Additives other than nutritional components such as carbohydrates and protein and fat may also be present in the particles. Additives that may be present include amino acids, molasses, coloring and dye ingredients, vitamins and minerals, nutraceuticals and pharmaceuticals and various processing aids such as talc and calcium carbonate. These additives may be added into the nutritional components or into the fat components. Carbohydrates include glucose, fructose, sucrose, and lactose. Minerals may be added, such as dicalcium phosphate, potassium citrate, ash, calcium, phosphorus, potassium, magnesium, sulfur, sodium, chloride, fluorine, iron, zinc, manganese, copper, cobalt, iodine, chromium, and/or selenium.

Vitamins and vitamin mixes may be added such as carotene, vitamin K, thiamin hydrochloride, riboflavin, niacin, pantothenic acid, choline chloride, folic acid, pyridoxine, biotin, B12, vitamin A, vitamin D, vitamin D3, vitamin E, ascorbic acid, and/or choline bitartrate

II. Models of Heart Failure with Preserved Ejection Fraction

The compositions of the disclosure may be administered to laboratory animals to induce heart failure. In some embodiments, the heart failure is heart failure with preserved ejection fraction.

In some embodiments, the dose of L-NAME administered to induce HFpEF is about 120 mg/kg body weight. In some embodiments, the dose of L-NAME is about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 290, 300, 310, 320, 330, 340, or 350 mg/kg (or any derivable range therein). In some embodiments, the dose is a daily dose.

Nearly half of all patients with heart failure have a normal ejection fraction (EF). The prevalence of this syndrome, termed heart failure with preserved ejection fraction (HFpEF), continues to increase in the developed world, likely because of the increasing prevalence of common risk factors, including older age, female sex, hypertension, metabolic syndrome, renal dysfunction and obesity.

Hypertension in particular is a strong risk factor; 80 to 90 percent of patients with HFpEF are hypertensive. Historically, HFpEF was termed diastolic heart failure; however, recent investigations suggest a more complex and heterogeneous pathophysiology. Ventricular diastolic and systolic reserve abnormalities, chronotropic incompetence, stiffening of ventricular tissue, atrial alterations, pulmonary hypertension, impaired vasodilation, and endothelial dysfunction are all implicated. Frequently, these abnormalities are noted only when the circulatory system is stressed.

At a cellular level, cardiac myocytes in patients with HFpEF are thicker and shorter than normal myocytes, and collagen content is increased. Recent histologic studies have shown reductions in myocardial capillary density that may contribute. At the organ level, affected individuals may have concentric remodeling with or without hypertrophy, although many people have normal ventricular geometry. Increases in myocyte stiffness are mediated in part by relative hypophosphorylation of the sarcomeric molecule titin, due to cyclic guanosine monophosphate (cGMP) deficiency thought to arise primarily as a consequence of increased nitroso-oxidative stress induced by comorbid conditions such as obesity, metabolic syndrome and aging. Cellular and tissue characteristics may become more pronounced as the disease progresses.

Most studies suggest that the rate of left ventricular (LV) pressure decay during isovolumic relaxation is prolonged, increasing LV and left atrial (LA) pressure, especially with elevated heart rates, as during exercise.

Normal ventricular filling is achieved in large part by ventricular suction, the early active component of diastole, which is generated by: intraventricular pressure gradients, mitral annular longitudinal motion, early diastolic LV “untwisting”, and elastic recoil induced by contraction to a smaller end systolic volume in the preceding contraction cycle.

Each of these four elements is impaired in patients with HFpEF, especially with stress, so filling becomes dependent on high LA pressure to actively push blood into the left ventricle, as opposed to the action of a normal left ventricle, which “pulls” blood in during early diastole. Passive LV end-diastolic stiffness (Eed) is quantified by the slope and position of the diastolic pressure-volume relationship. Eed increases with normal aging, but this increase is exaggerated in individuals with HFpEF in most, but not all studies.

Although systolic function is relatively preserved, individuals with HFpEF typically exhibit subtle abnormalities in systolic performance, which become more dramatic during exercise. Limited stroke volume reserve and chronotropic incompetence markedly limit cardiac output in response to exercise. Mechanical dyssynchrony is common even though electrical dyssynchrony is not. Atrial fibrillation is extremely common in HFpEF (seen at some point in two-thirds of patients) and poorly tolerated because of the importance of LA contractile function in maintaining adequate LV chamber filling.

Pulmonary hypertension is common in patients with HFpEF. Increased LA pressure adds in series with increased resistive and pulsatile pulmonary arterial loading to increase RV afterload. This then leads to RV dysfunction, which seems to be tightly correlated with the development of atrial fibrillation.

Increased RV and LA size and subsequent increases in total cardiac volume can lead to pericardial restraint, preventing additional preload recruitment during exercise or saline loading and contributing to elevation in filling pressures and cardiac output plateau.

The methods described herein for inducing heart failure such as HFpEF induce phenotypes in the laboratory animal that are characteristic of heart failure with preserved ejection fraction and/or occur in conjunction with heart failure such as obesity, glucose intolerance, hypertension, normal or near normal ejection fraction, diastolic dysfunction, impaired systolic function with global longitudinal strain reduction, exercise intolerance, cardiomyocyte hypertrophy, reduced cardiomyocyte contractility and relaxation, increased cardiac fibrosis, increased vascular stiffness, impaired coronary flow reserve, increased atrial fibrillation episodes and lung congestion.

### III. Assays

Method aspects of the disclosure also include the determination of certain perameters and/or the performance of certain assays. Exemplary assays are described below.

A. Echocardiography

An echocardiogram, often referred to as a cardiac echo or simply an echo, is a sonogram of the heart. Echocardiography uses standard two-dimensional, three-dimensional, and Doppler ultrasound to create images of the heart. Echocardiography has become routinely used in the diagnosis, management, and follow-up of patients with any suspected or known heart diseases. It is one of the most widely used diagnostic tests in cardiology. It can provide a wealth of helpful information, including the size and shape of the heart (internal chamber size quantification), pumping capacity, and the location and extent of any tissue damage. An echocardiogram can also give physicians other estimates of heart function, such as a calculation of the cardiac output, ejection fraction, and diastolic function (how well the heart relaxes).

Echocardiography can help detect cardiomyopathies, such as hypertrophic cardiomyopathy, dilated cardiomyopathy, and many others. The use of stress echocardiography may also help determine whether any chest pain or associated symptoms are related to heart disease. The biggest advantage to echocardiography is that it is not invasive (does not involve breaking the skin or entering body cavities) and has no known risks or side effects.

An echocardiogram can not only create ultrasound images of heart structures, but it can also produce accurate assessment of the blood flowing through the heart by Doppler echocardiography, using pulsed- or continuous-wave Doppler ultrasound. This allows assessment of both normal and abnormal blood flow through the heart. Color Doppler, as well as spectral Doppler, is used to visualize any abnormal communications between the left and right sides of the heart, any leaking of blood through the valves (valvular regurgitation), and estimate how well the valves open (or do not open in the case of valvular stenosis). The Doppler technique can also be used for tissue motion and velocity measurement, by tissue Doppler echocardiography. Finally, Doppler echography can be also used to measured coronary flow reserve, defined as the maximum increase in blood flow through the coronary arteries above the normal resting volume.

B. Magnetic Resonance Imaging

Magnetic resonance imaging (MRI) is a medical imaging technique used in radiology to form pictures of the anatomy and the physiological processes of the body in both health and disease. MRI scanners use strong magnetic fields, radio waves, and field gradients to generate images of the organs in the body. MRI does not involve x-rays, which distinguishes it from computed tomography (CT or CAT). MRI often may yield different diagnostic information compared with CT. MRI is based upon the science of nuclear magnetic resonance (NMR). Certain atomic nuclei are able to absorb and emit radio frequency energy when placed in an external magnetic field. In clinical and research MRI, hydrogen atoms are most-often used to generate a detectable radio-frequency signal that is received by antennas in close proximity to the anatomy being examined. Hydrogen atoms exist naturally in people and other biological organisms in abundance, particularly in water and fat. For this reason, most MRI scans essentially map the location of water and fat in the body. Pulses of radio waves excite the nuclear spin energy transition, and magnetic field gradients localize the signal in space. By varying the parameters of the pulse sequence, different contrasts may be generated between tissues based on the relaxation properties of the hydrogen atoms therein.

While Mill is most prominently used in diagnostic medicine and biomedical research, it also may be used to form images of non-living objects. MRI scans are capable of producing a variety of chemical and physical data, in addition to detailed spatial images. MRI is widely used in hospitals and clinics for medical diagnosis, staging of disease and follow-up without exposing the body to ionizing radiation.

Cardiac MRI is complementary to other imaging techniques, such as echocardiography, cardiac CT, and nuclear medicine. Its applications include assessment of myocardial ischemia and viability, cardiomyopathies, myocarditis, iron overload, vascular diseases, and congenital heart disease.

C. Computedtomography (CT)

A CT scan makes use of computer-processed combinations of many X-ray images taken from different angles to produce cross-sectional (tomographic) images (virtual “slices”) of specific areas of a scanned object, allowing the user to see inside the object without cutting. Other terms include computed axial tomography (CAT scan) and computer aided tomography. Digital geometry processing is used to generate a three-dimensional image of the inside of the object from a large series of two-dimensional radiographic images taken around a single axis of rotation. Medical imaging is the most common application of X-ray CT. Its cross-sectional images are used for diagnostic and therapeutic purposes in various medical disciplines.

The term “computed tomography” (CT) is often used to refer to X-ray CT, because it is the most commonly known form. But, many other types of CT exist, such as positron emission tomography (PET) and single-photon emission computed tomography (SPECT). X-ray tomography is one form of radiography, along with many other forms of tomographic and non-tomographic radiography.

CT produces a volume of data that can be manipulated in order to demonstrate various bodily structures based on their ability to block the X-ray beam. Although, historically, the images generated were in the axial or transverse plane, perpendicular to the long axis of the body, modern scanners allow this volume of data to be reformatted in various planes or even as volumetric (3D) representations of structures.

With the advent of subsecond rotation combined with multi-slice CT (up to 320-slices), high resolution and high speed can be obtained at the same time, allowing excellent imaging of the coronary arteries (cardiac CT angiography).

D. Single-Photon Emission Computed Tomography (SPECT)

Single-photon emission computed tomography (SPECT, or less commonly, SPET) is a nuclear medicine tomographic imaging technique using gamma rays. It is very similar to conventional nuclear medicine planar imaging using a gamma camera (that is, scintigraphy). However, it is able to provide true 3D information. This information is typically presented as cross-sectional slices through the patient, but can be freely reformatted or manipulated as required.

The technique requires delivery of a gamma-emitting radioisotope (a radionuclide) into the patient, normally through injection into the bloodstream. On occasion, the radioisotope is a simple soluble dissolved ion, such as an isotope of gallium(III). Most of the time, though, a marker radioisotope is attached to a specific ligand to create a radioligand, whose properties bind it to certain types of tissues. This marriage allows the combination of ligand and radiopharmaceutical to be carried and bound to a place of interest in the body, where the ligand concentration is seen by a gamma camera.

SPECT can be used to complement any gamma imaging study, where a true 3D representation can be helpful, e.g., tumor imaging, infection (leukocyte) imaging, thyroid imaging or bone scintigraphy. Because SPECT permits accurate localisation in 3D space, it can be used to provide information about localised function in internal organs, such as functional cardiac or brain imaging.

Myocardial perfusion imaging (MPI) is a form of functional cardiac imaging, used for the diagnosis of ischemic heart disease. The underlying principle is that under conditions of stress, diseased myocardium receives less blood flow than normal myocardium. MPI is one of several types of cardiac stress test. A cardiac specific radiopharmaceutical is administered, e.g., 99mTc-tetrofosmin (Myoview, GE healthcare), 99mTc-sestamibi (Cardiolite, Bristol-Myers Squibb) or technetium-99m. Following this, the heart rate is raised to induce myocardial stress, either by exercise on a treadmill or pharmacologically with adenosine, dobutamine, or dipyridamole (aminophylline can be used to reverse the effects of dipyridamole). SPECT imaging performed after stress reveals the distribution of the radiopharmaceutical, and therefore the relative blood flow to the different regions of the myocardium. Diagnosis is made by comparing stress images to a further set of images obtained at rest which are normally acquired prior to the stress images. MPI has been demonstrated to have an overall accuracy of about 83% (sensitivity: 85%; specificity: 72%), [3] and is comparable with (or better than) other non-invasive tests for ischemic heart disease.

E. Nuclear Magnetic Resonance Spectroscopy

Nuclear magnetic resonance spectroscopy, most commonly known as NMR spectroscopy, is a research technique that exploits the magnetic properties of certain atomic nuclei. This type of spectroscopy determines the physical and chemical properties of atoms or the molecules in which they are contained. It relies on the phenomenon of nuclear magnetic resonance and can provide detailed information about the structure, dynamics, reaction state, and chemical environment of molecules. The intramolecular magnetic field around an atom in a molecule changes the resonance frequency, thus giving access to details of the electronic structure of a molecule and its individual functional groups.

Most frequently, NMR spectroscopy is used by chemists and biochemists to investigate the properties of organic molecules, although it is applicable to any kind of sample that contains nuclei possessing spin. Suitable samples range from small compounds analyzed with 1-dimensional proton or carbon-13 NMR spectroscopy to large proteins or nucleic acids using 3 or 4-dimensional techniques. The impact of NMR spectroscopy on the sciences has been substantial because of the range of information and the diversity of samples, including solutions and solids.

NMR spectra are unique, well-resolved, analytically tractable and often highly predictable for small molecules. Thus, in organic chemistry practice, NMR analysis is used to confirm the identity of a substance. Different functional groups are obviously distinguishable, and identical functional groups with differing neighboring substituents still give distinguishable signals. NMR has largely replaced traditional wet chemistry tests such as color reagents or typical chromatography for identification. A disadvantage is that a relatively large amount, 2-50 mg, of a purified substance is required, although it may be recovered through a workup. Preferably, the sample should be dissolved in a solvent, because NMR analysis of solids requires a dedicated MAS machine and may not give equally well-resolved spectra. The timescale of NMR is relatively long, and thus it is not suitable for observing fast phenomena, producing only an averaged spectrum. Although large amounts of impurities do show on an NMR spectrum, better methods exist for detecting impurities, as NMR is inherently not very sensitive—though at higher frequencies, sensitivity narrows.

F. Cardiac Catheterization and Coronary Angiography

Cardiac catheterization and coronary angiography are semi-invasive methods of studying the heart and the blood vessels that supply the heart (coronary arteries) without doing surgery. Cardiac catheterization is used extensively for the diagnosis and treatment of various heart disorders. Cardiac catheterization can be used to measure how much blood the heart pumps out per minute (cardiac output), to detect birth defects of the heart, and to detect and biopsy tumors affecting the heart (for example, a myxoma). This procedure is the only way to directly measure the pressure of blood in each chamber of the heart and in the major blood vessels going from the heart to the lungs.

In cardiac catheterization, a thin catheter (a small, flexible, hollow plastic tube) is inserted into an artery or vein in the neck, arm, or groin/upper thigh through a puncture made with a needle. A local anesthetic can be given to numb the insertion site. The catheter is then threaded through the major blood vessels and into the chambers of the heart.

Various small instruments can be advanced through the tube to the tip of the catheter. They include instruments to measure the pressure of blood in each heart chamber and in blood vessels connected to the heart, to view or take ultrasound images of the interior of blood vessels, to take blood samples from different parts of the heart, or to remove a tissue sample from inside the heart for examination under a microscope (biopsy).

In an angiography, a catheter is used to inject a radiopaque contrast agent into blood vessels so that they can be seen on x-rays. In a ventriculography, a catheter is used to inject a radiopaque contrast agent into one or more heart chambers so that they can be seen on x-rays. In a valvuloplasty, a catheter is used to widen a narrowed heart valve opening.

Ventriculography is a type of angiography in which x-rays are taken as a radiopaque contrast agent is injected into the left or right ventricle of the heart through a catheter. It is done during cardiac catheterization. With this procedure, doctors can see the motion of the left or right ventricle and can thus evaluate the pumping ability of the heart. Based on the heart's pumping ability, doctors can calculate the ejection fraction (the percentage of blood pumped out by the left ventricle with each heartbeat). Evaluation of the heart's pumping helps determine how much of the heart has been damaged.

If an artery is used for catheter insertion, the puncture site must be steadily compressed for 10 to 20 minutes after all the instruments are removed. Compression prevents bleeding and bruise formation. However, bleeding occasionally occurs at the puncture site, leaving a large bruise that can persist for weeks but that almost always goes away on its own.

Because inserting a catheter into the heart may cause abnormal heart rhythms, the heart is monitored with electrocardiography (ECG). Usually, doctors can correct an abnormal rhythm by moving the catheter to another position. If this maneuver does not help, the catheter is removed. Very rarely, the heart wall is damaged or punctured when a catheter is inserted, and immediate surgical repair may be required.

Cardiac catheterization may be done on the right or left side of the heart. Catheterization of the right side of the heart is done to obtain information about the heart chambers on the right side (right atrium and right ventricle) and the tricuspid valve (located between these two chambers). The right atrium receives oxygen-depleted blood from the veins of the body, and the right ventricle pumps the blood into the lungs, where blood takes up oxygen and drops off carbon dioxide. In this procedure, the catheter is inserted into a vein, usually in the neck or the groin. Pulmonary artery catheterization, in which a balloon at the catheter's tip is passed through the right atrium and ventricle and lodged in the pulmonary artery, is sometimes done during certain major operations and in intensive care units. Right-side catheterization is used to detect and quantify abnormal connections between the right and left sides of the heart. Doctors usually use right-side catheterization when evaluating people for heart transplantation or for the placement of a mechanical device to help pump blood. Catheterization of the left side of the heart is done to obtain information about the heart chambers on the left side (left atrium and left ventricle), which are the mitral valve (located between the left atrium and left ventricle), and the aortic valve (located between the left ventricle and the aorta). The left atrium receives oxygen-rich blood from the lungs, and the left ventricle pumps the blood into the rest of the body. This procedure is usually combined with coronary angiography to obtain information about the coronary arteries. For catheterization of the left side of the heart, the catheter is inserted into an artery, usually in an arm or the groin.

Coronary angiography provides information about the coronary arteries, which supply the heart with oxygen-rich blood. Coronary angiography is similar to catheterization of the left side of the heart because the coronary arteries branch off the aorta just after it leaves the left side of the heart (see Blood Supply of the Heart). The two procedures are almost always done at the same time. After injecting a local anesthetic, a doctor inserts a thin catheter into an artery through an incision in an arm or the groin. The catheter is threaded toward the heart, then into the coronary arteries. During insertion, the doctor uses fluoroscopy (a continuous x-ray procedure) to observe the progress of the catheter as it is threaded into place. After the catheter tip is in place, a radiopaque contrast agent (dye), which can be seen on x-rays, is injected through the catheter into the coronary arteries, and the outline of the arteries appears on a video screen and is recorded.

Doctors use these images to detect blockages (coronary artery disease) or spasms of the coronary arteries. Images can help determine whether angioplasty (opening the blockage with a small balloon inserted through the catheters) and metal stent placement (to keep the coronary artery open) is needed or whether coronary artery bypass surgery should be done to get blood past the area of blockage. Miniature ultrasound transducers on the end of coronary artery catheters can produce images of coronary vessel walls and show blood flow. This technique is being increasingly used at the same time as coronary angiography. More recently, a related procedure called optical coherence tomography is used to determine temperature of plaques on the artery walls and can help to determine if the plaques are at high risk of breaking free and causing a heart attack.

G. Hemodynamic Studies

Hemodynamics or hemodynamics is the dynamics of blood flow. The circulatory system is controlled by homeostatic mechanisms, much as hydraulic circuits are controlled by control systems. Hemodynamic response continuously monitors and adjusts to conditions in the body and its environment. Thus hemodynamics explains the physical laws that govern the flow of blood in the blood vessels. Hemodynamic analysis may be performed on the laboratory animals of the disclosure or tissues thereof. The analysis may include a measurement of the viscosity of plasma, the osmotic pressure of plasma, the number or quality of red blood cells, the cardiac output and flow rate, the blood pressure, such as a determination of the mean arterial pressure, the diastolic blood pressure, and the systolic blood pressure, blood flow velocity, vascular resistance, blood turbulence, wall tension, venous capacitance, and heart rate or blood pressure monitoring over time.

H. Vascular Stiffness

Laboratory animals such as rodent models are increasingly used to study the development and progression of arterial stiffness. Both the non-invasive Doppler derived Pulse Wave Velocity (PWV) and the invasively determined arterial elastance index (EaI) have been used to assess arterial stiffness in the laboratory. To determine PWV, the laboratory animal can be anesthetized with isoflurane. The animal can then be situated in the supine position on a controlled heating pad to maintain a body temperature of 37° C. and EKG limb electrodes can then be placed. An Acuson Sequoia C512 Ultrasound System with a 15 MHz linear array transducer and color-flow Doppler capabilities (Siemens Medical) can be used to scan the carotid and iliac arteries. Color-flow Doppler can be employed to help locate the arteries and guide placement of the sample gate for obtaining pulse wave forms. The probe can be directed parallel to blood flow. EKG and Doppler signals can then be recorded simultaneously at a sweep speed of 200 mm/sec for several cardiac cycles, and the data were stored for subsequent off-line analysis. At the end of the study, the distance measured in mm (D) between the points of probe applanation over the carotid and iliac arteries can be measured using a tape measure. The time intervals (measured in msec) between the R-wave of the EKG to the foot of the Doppler carotid and iliac waveforms can be averaged over multiple cardiac cycles, and the pulse-transit time from the carotid to iliac arteries (T) can be calculated by subtracting the mean R-carotid foot time interval from the mean R-iliac foot time interval. PWV can be then calculated as: PWV=Distance (D)/Time (T), where D is the distance in mm from carotid to iliac applanation sites and T=(R to iliac foot)−(R to carotid foot) in msec.

I. Other Assays

Other assays that may be performed in the methods of the disclosure include, for example, measurement of blood glucose levels, exercise tolerance tests, coronary flow reserve measurement, histological evaluation of tissues and their morphology, such as cardiac morphology, and evaluation of skeletal muscle force.

J. Drug development

In some embodiments, the laboratory animals and methods of the disclosure comprise drug development techniques such as administering a compound to a HFpEF laboratory animal or tissue thereof and monitoring certain parameters, such as those described in the disclosure of the application. In some embodiments, the methods comprise the performance of pharmacokinetic studies, such as the measurement of the absorption, bio-distribution, metabolism, escretion, toxicity, and/or efficacy of a compound, protein, or nucleic acid molecule.

### EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of particular embodiments, are exemplary, and are not intended as limitations on the scope of the invention. Changes therein and other uses which are encompassed within the spirit of the invention as defined by the scope of the claims will occur to those skilled in the art.

### Example 1: Model of Heart Failure with Preserved Ejection Fraction

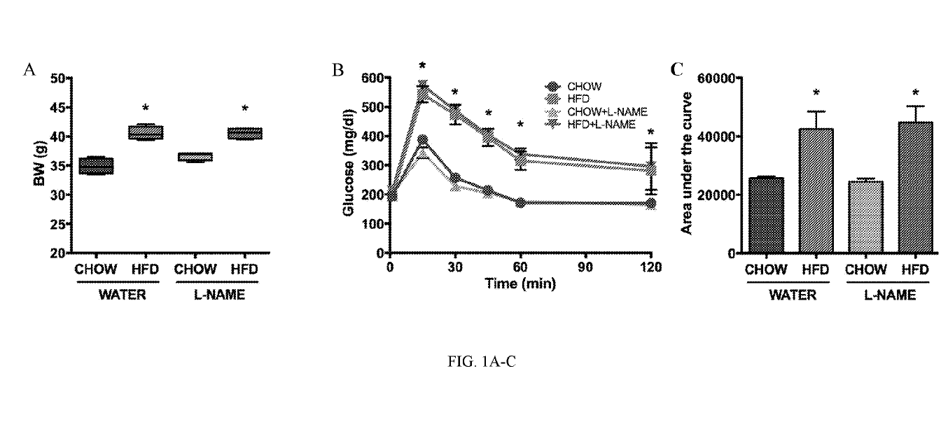

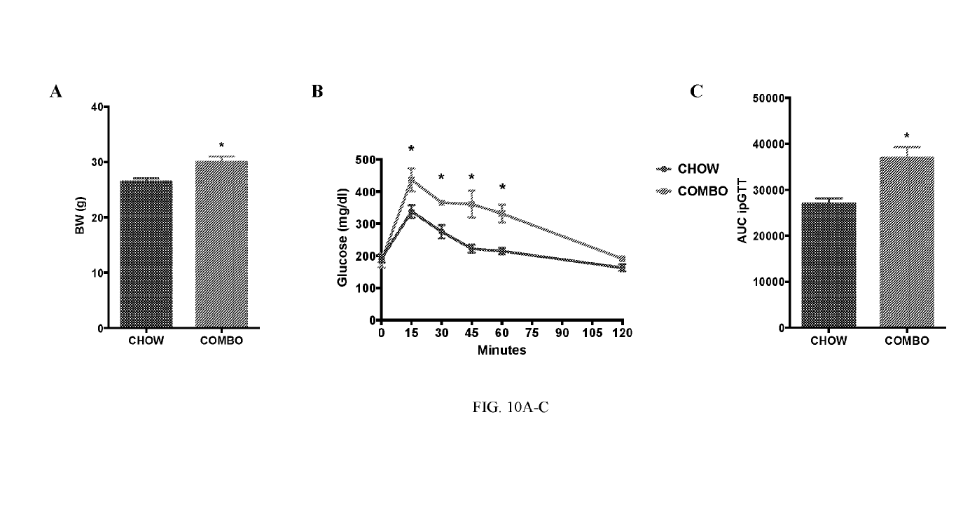

Mice were fed a high fat diet (HFD—Table 1, 5.24 total kcal/gm) in conjunction with 0.5 g/L of L-NAME in the drinking water (FIGS. 1-9) or formulated into the HFD food product during the manufacturing stage (FIGS. 10-13, “combo diet”). Based on mouse mean daily water intake, this level of L-NAME in the drinking water provided about 0.07% w/w of the animal's diet. In some embodiments, the dose of L-NAME is about 120 mg/kg body weight.

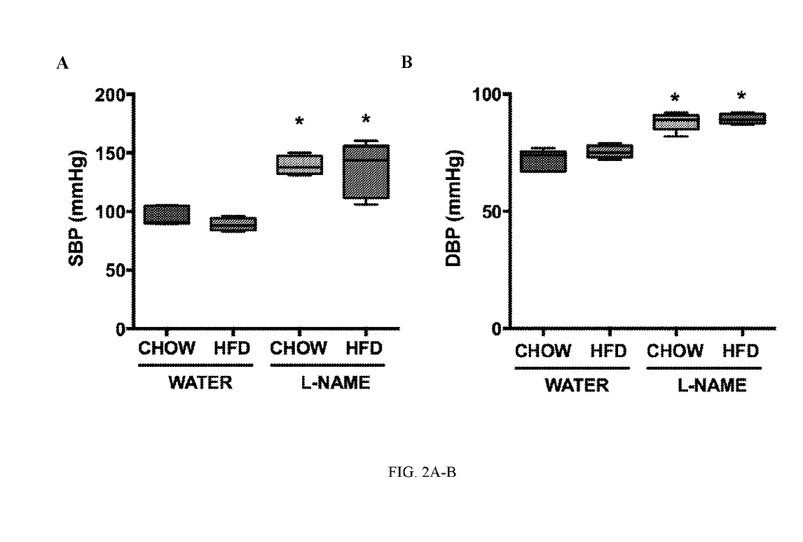

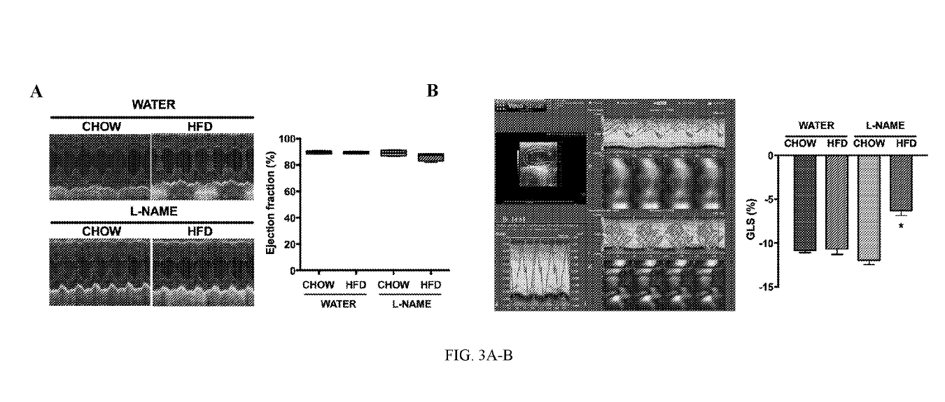

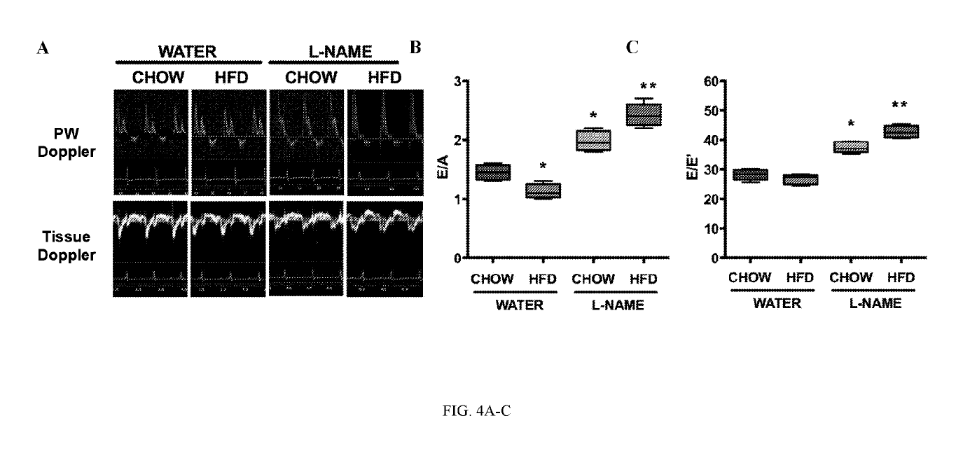

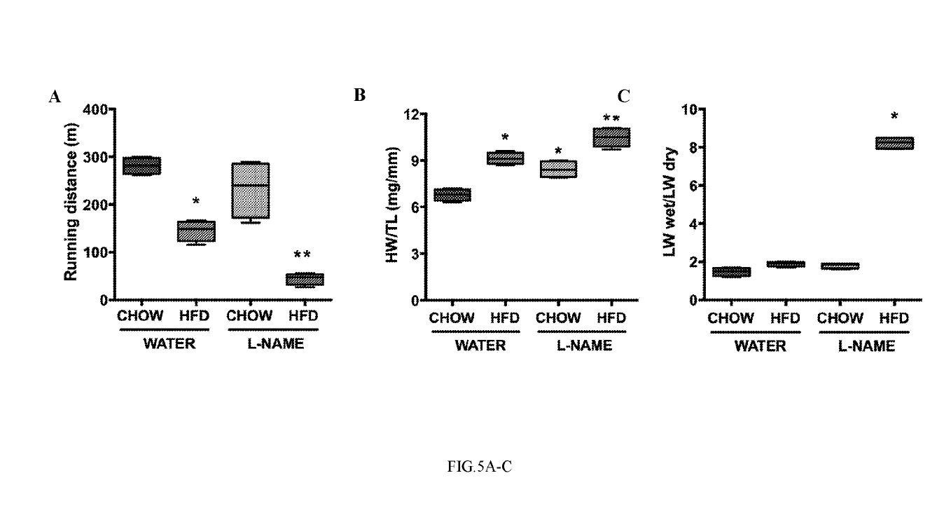

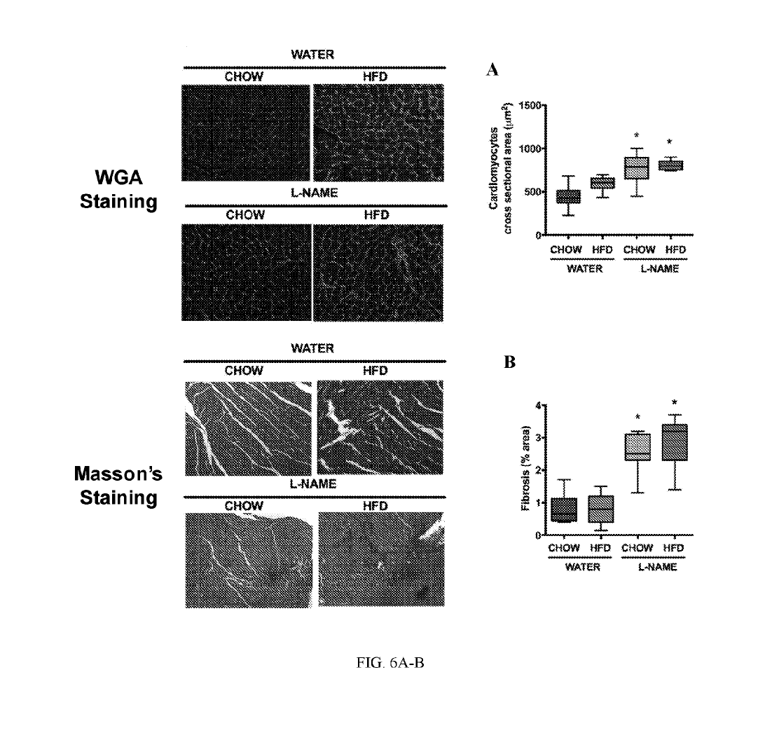

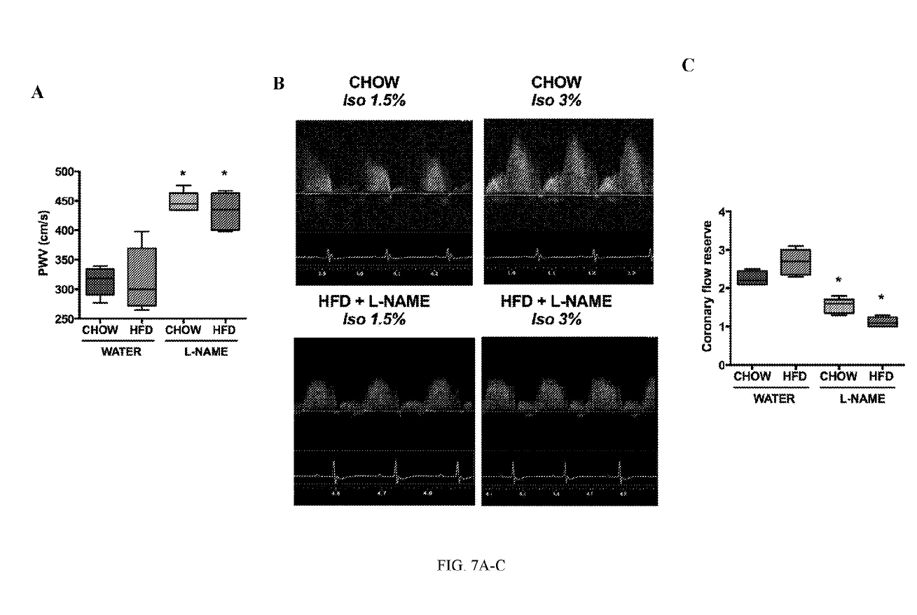

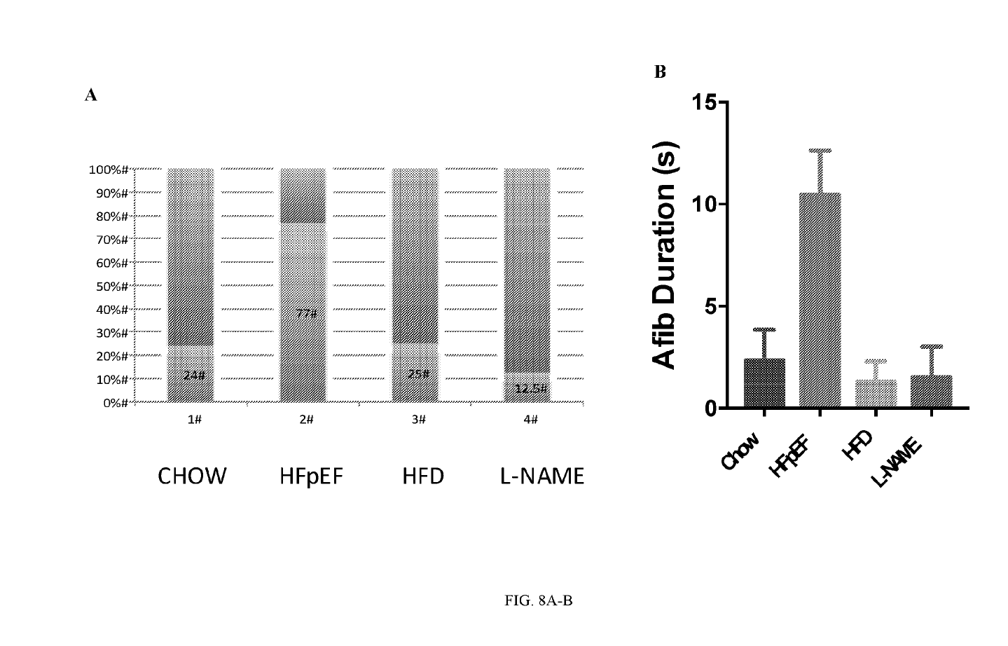

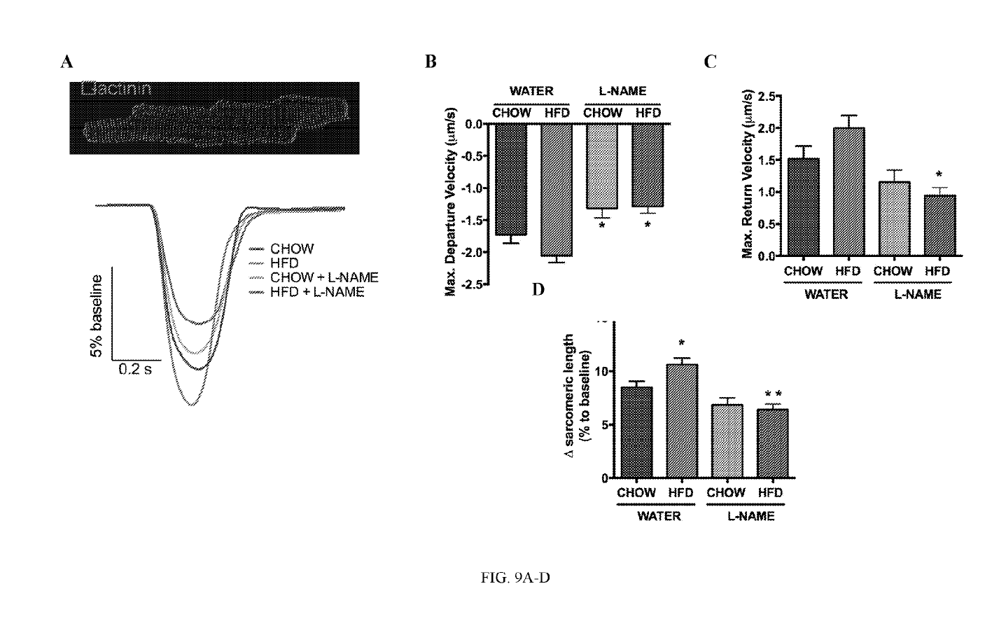

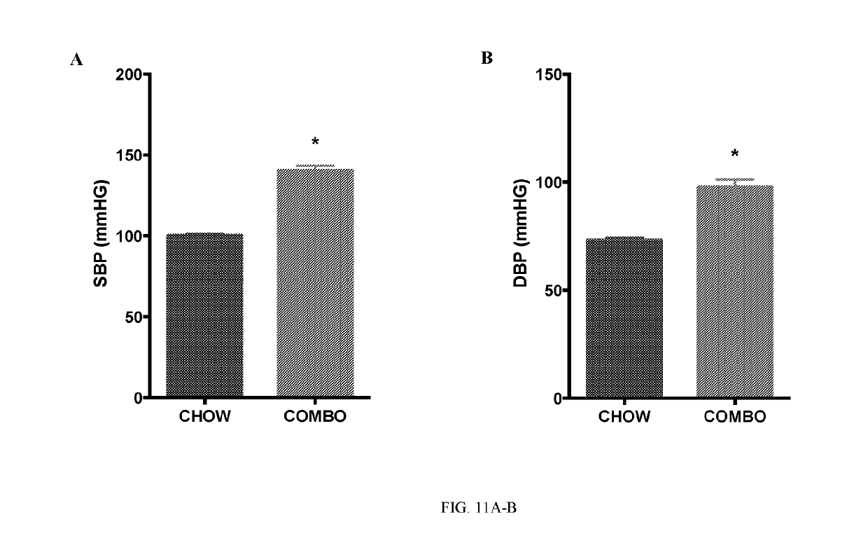

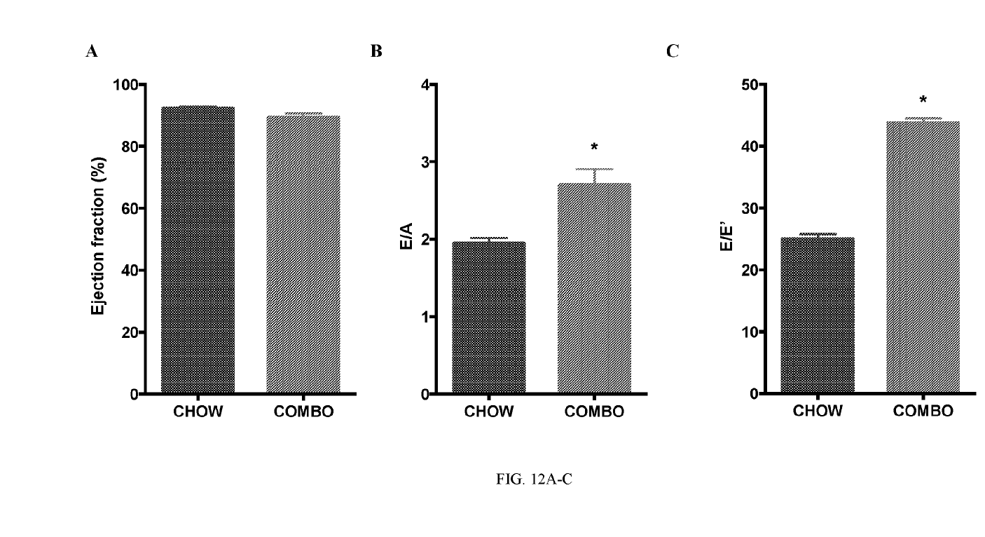

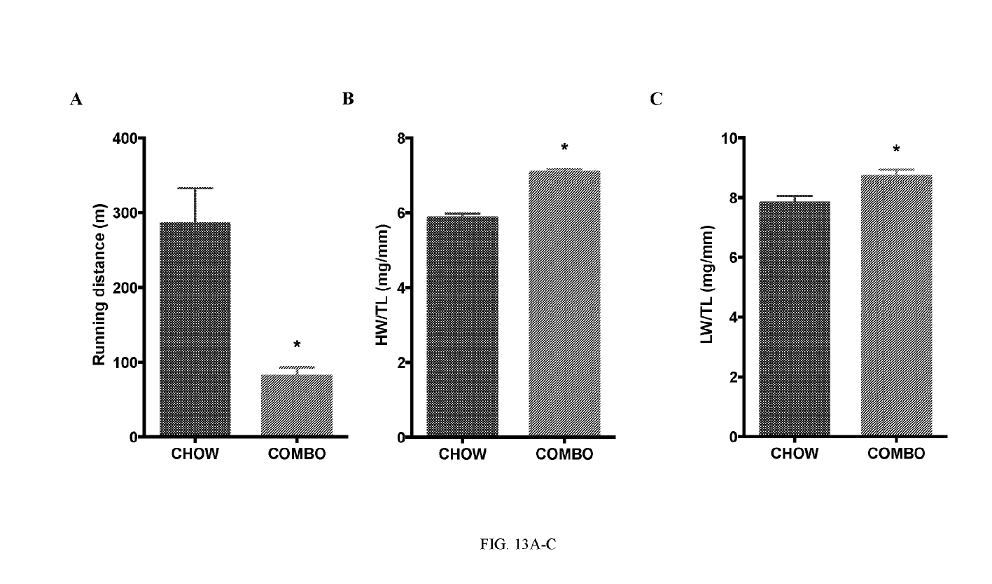

As shown in the figures, HFpEF mice (mice fed HFD+L-Name) exhibit a HFpEF phenotype. Specifically, these mice exhibited obesity (FIGS. 1A, 10A) and glucose intolerance (FIGS. 1B, 1C, 10B, 10C), increased systolic blood pressure (FIGS. 2A and 11A), increased diastolic blood pressure (FIGS. 2B and 11B), have preserved ejection fraction (EF %) (FIGS. 3A and 12A) with impaired systolic function (FIG. 3B), impaired diastolic function (FIGS. 4A-C and 12B-C), exercise intolerance (FIGS. 5A and 13A), cardiac hypertrophy (FIGS. 5B and 13B), signs of heart failure (FIGS. 5C and 13C), cardiomyocyte hypertrophy (FIG. 6A), increased fibrosis (FIG. 6B), increased vascular stiffness (FIG. 7A), impaired coronary flow reserve (CFR) (FIG. 7B-C), are more susceptible to induction of atrial fibrillation (Afib) (FIG. 8A) with an increase of the duration of episodes (FIG. 8B), impaired contractility of isolated cardiomyocytes (FIG. 9A-B), and reduced relaxation (FIGS. 9A, C, and D). In conclusion, mice fed a high fat diet with L-Name, either added to the drinking water or incorporated into the food, exhibit a HFpEF phenotype and are useful for modeling this condition.

TABLE 1
High fat diet (HFD)

| | gm % | kcal % |
|---|---|---|
| Protein | 26.2 | 20 |
| Carbohydrate | 26.3 | 20 |
| Fat | 34.9 | 60 |
| Ingredient | gm | kcal |
| Casein, 80 Mesh | 200 | 800 |
| L-Cystine | 3 | 12 |
| Corn Starch | 0 | 0 |
| Maltodextrin 10 | 125 | 500 |
| Sucrose | 68.8 | 275.2 |
| Cellulose, BW200 | 50 | 0 |
| Soybean Oil | 25 | 225 |
| Lard* | 245 | 2205 |
| Mineral Mix, S10026 | 10 | 0 |
| DiCalcium Phosphate | 13 | 0 |
| Calcium Carbonate | 5.5 | 0 |
| Potassium Citrate, 1 | 16.5 | 0 |
| H2O | | |
| Vitamin Mix, V10001 | 10 | 40 |
| Choline Bitartrate | 2 | 0 |
| FD&C Blue Dye #1 | 0.05 | 0 |
| Total | 773.85 | 4057 |

*Typical analysis of cholesterol in lard = 0.95 mg/gram.
Cholesterol (mg)/4057 kcal = 232.8
Cholesterol (mg)/kg = 300.8

The “Chow” diet used to collect the data of the examples has the following composition:

TABLE 2
Chow diet
MACRONUTRIENTS

| Crude Protein | 16.4% | |
|---|---|---|
| Fat (ether extract)a | 4.0% | |
| Carbohydrate (available)b | 48.5% | |
| Crude Fiber | 3.3% | |
| Neutral Detergent Fiberc | 15.2% | |
| Ash | 4.9% | |
| Energy Densityd | 3.0 (12.6) | kcal/g (kJ/g) |

| | |
|---|---|
| Calories from Protein | 22% |
| Calories from Fat | 12% |
| Calories from Carbohydrate | 66% |

MINERALS

| Calcium | 1.0% | |
|---|---|---|
| Phosphorus | 0.7% | |
| Non-Phytate Phosphorus | 0.4% | |
| Sodium | 0.2% | |
| Potassium | 0.6% | |
| Chloride | 0.4% | |
| Magnesium | 0.2% | |
| Zinc | 70 | mg/kg |
| Manganese | 100 | mg/kg |
| Copper | 15 | mg/kg |
| Iodine | 6 | mg/kg |
| Iron | 200 | mg/kg |
| Selenium | 0.23 | mg/kg |

AMINO ACIDS

| | |
|---|---|
| Aspartic Acid | 1.0% |
| Glutamic Acid | 3.3% |
| Alanine | 0.9% |
| Glycine | 0.7% |
| Threonine | 0.6% |
| Proline | 1.5% |
| Serine | 0.8% |
| Leucine | 1.9% |
| Isoleucine | 0.7% |
| Valine | 0.8% |
| Phenylalanine | 0.9% |
| Tyrosine | 0.5% |
| Methionine | 0.3% |
| Cystine | 0.3% |
| Lysine | 0.8% |
| Histidine | 0.4% |
| Arginine | 0.8% |
| Tryptophan | 0.2% |

VITAMINS

| | | |
|---|---|---|
| Vitamin A^{e,f} | 15.0 | IU/g |
| Vitamin D₃^{e,g} | 1.5 | IU/g |
| Vitamin E | 110 | IU/kg |
| Vitamin K₃ (menadione) | 50 | mg/kg |
| Vitamin B₁ (thiamin) | 17 | mg/kg |
| Vitamin B₂ (riboflavin) | 15 | mg/kg |
| Niacin (nicotinic acid) | 75 | mg/kg |
| Vitamin B₆ (pyridoxine) | 18 | mg/kg |
| Pantothenic Acid | 33 | mg/kg |
| Vitamin B₁₂ (cyanocobalamin) | 0.08 | mg/kg |
| Biotin | 0.40 | mg/kg |
| Folate | 4 | mg/kg |
| Choline | 1030 | mg/kg |

FATTY ACIDS

| | |
|---|---|
| C16:0 Palmitic | 0.5% |
| C18:0 Stearic | 0.1% |
| C18:1ω9 Oleic | 0.7% |
| C18:2ω6 Linoleic | 2.0% |
| C18:3ω3 Linolenic | 0.1% |
| Total Saturated | 0.6% |
| Total Monounsaturated | 0.7% |
| Total Polyunsaturated | 2.1% |

OTHER

| | | |
|---|---|---|
| Cholesterol | -- | mg/kg |

^{a}Ether extract is used to measure fat in pelleted diets, while an acid hydrolysis method is required to recover fat in extruded diets. Compared to ether extract, the fat value for acid hydrolysis will be approximately 1% point higher.
^{b}Carbohydrate (available) is calculated by subtracting neutral detergent fiber from total carbohydrates.
^{c}Neutral detergent fiber is an estimate of insoluble fiber, including cellulose, hemicellulose, and lignin. Crude fiber methodology underestimates total fiber.
^{d}Energy density is a calculated estimate of metabolizable energy based on the Atwater factors assigning 4 kcal/g to protein, 9 kcal/g to fat, and 4 kcal/g to available carbohydrate.
^{e}Indicates added amount but does not account for contribution from other ingredients.
^{f}1 IU vitamin A = 0.3 μg retinol
^{g}1 IU vitamin D = 25 ng cholecalciferol

An example of a feed composition comprising L-NAME is provided in Table 3.

TABLE 3
Feed Composition with L-NAME

| Ingredient | gm | % w/w |
|---|---|---|
| Casein, 80 Mesh | 200 | 25.82 |
| L-Cystine | 3 | 0.39 |
| Corn Starch | 0 | 0.00 |
| Maltodextrin 10 | 125 | 16.14 |
| Sucrose | 68.8 | 8.88 |
| Cellulose, BW200 | 50 | 6.45 |
| Soybean Oil | 25 | 3.23 |
| Lard* | 245 | 31.64 |
| Mineral Mix, S10026 | 10 | 1.29 |
| DiCalcium Phosphate | 13 | 1.68 |
| Calcium Carbonate | 5.5 | 0.71 |
| Potassium Citrate, 1 | 16.5 | 2.13 |
| H2O | | |
| Vitamin Mix, V10001 | 10 | 1.29 |
| Choline Bitartrate | 2 | 0.26 |
| FD&C Blue Dye #1 0 | 0.05 | 0.006 |
| L-NAME | 0.58 | 0.07 |
| Total | 774.43 | |

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus-or step-plus-function limitations, unless such a limitation is explicitly recited in a given claim using the phrase(s) “means for” or “step for,” respectively.

1. A composition comprising 20-60% w/w of fat combined with a nitric oxide synthase inhibitor.

2. The composition of claim 1, wherein the nitric oxide synthase inhibitor is L-NAME or a salt thereof.

3. The composition of claim 1 or 2, wherein the composition comprises about 0.05-0.1% w/w of nitric oxide synthase inhibitor.

4. The composition of any one of claims 1-3, wherein the composition comprises 27-37% w/w of fat combined with 0.05-0.1% w/w L-NAME or a salt thereof.

5. The composition of claim 1 or 4, wherein the composition comprises at least 0.01% w/w cholesterol.

6. The composition of claim 5, wherein the composition comprises 0.01-0.05% w/w cholesterol.

7. The composition of any one of claims 1-6, wherein the composition further comprises 10-30% w/w protein.

8. The composition of any one of claims 1-7, wherein the composition further comprises 10-30% w/w carbohydrate.

9. The composition of any one of claims, wherein the composition further comprises minerals and/or vitamins.

10. The composition of any one of claims 1-9, wherein the composition is formulated for oral ingestion.

11. The composition of claim 10, wherein the composition is formulated for animal consumption.

12. The composition of any one of claims 1-11, wherein the composition comprises:
0.05-0.1% w/w of L-NAME or a salt thereof;
27-37% w/w of fat;
10-30% w/w protein; and
10-30% w/w carbohydrate.

13. The composition of any one of claims 1-12, wherein the protein comprises Casein and/or amino acids.

14. The composition of claim 13, wherein the amino acid is L-Cystine.

15. The composition of any one of claims 1-14, wherein the composition further comprises a starch.

16. The composition of any one of claims 1-15, wherein the composition comprises corn starch, maltodextrin, sucrose, and/or cellulose.

17. The composition of any one of claims 1-16, wherein the composition comprises soybean oil and/or lard.

18. The composition of claim 17, wherein the lard comprises 0.5-1% w/w of cholesterol.

19. The composition of any one of claims 1-18, wherein the composition further comprises di-calcium phosphate, calcium carbonate, potassium citrate, and/or choline bitartrate.

20. A method for inducing heart failure with preserved ejection fraction in an experimental laboratory animal, the method comprising administering the composition of any one of claims 1-19 to the laboratory animal.

21. A method for inducing heart failure with preserved ejection fraction in an experimental laboratory animal, the method comprising administering a composition comprising 20-60% w/w of fat and a composition comprising a nitric oxide synthase inhibitor to the laboratory animal.

22. The method of claim 21, wherein the nitric oxide synthase inhibitor is L-NAME w/w or a salt thereof.

23. The method of claim 21 or 22, wherein the composition comprises about 0.05-0.1% w/w of nitric oxide synthase inhibitor.

24. The method of any one of claims 21-23, wherein the nitric oxide synthase inhibitor is administered to the animal in a food product.

25. The method of any one of claims 21-24, wherein the nitric oxide synthase inhibitor is admistered to the animal in a solution.

26. The method of claim 25, wherein the nitric oxide synthase inhibitor is administered to the animal in the drinking water.

27. The method of any one of claims 20-26, wherein the heart failure is characterized by normal or near normal systolic function.

28. The method of claim any one of claims 20-27, wherein the heart failure is characterized by obesity and/or insulin resistance.

29. The method any one of claims 20-28, wherein the heart failure is characterized by hypertension.

30. The method of claim 29, wherein the systolic and/or diastolic blood pressure is increased by at least 10 points.

31. The method of any one of claims 20-30, wherein the heart failure is characterized by normal or near normal ejection fraction, diastolic dysfunction, impaired systolic function with global longitudinal strain reduction, exercise intolerance, cardiomyocyte hypertrophy, reduced cardiomyocyte contractility and relaxation, increased cardiac fibrosis, increased vascular stiffness, impaired coronary flow reserve, increased atrial fibrillation episodes and lung congestion.

32. The method of any one of claims 20-31, wherein the animal is a mouse, a rat, a rabbit, a dog, a cat, a pig, a horse, a sheep, a goat or a monkey and non-human primate.

33. The method of claim 32, wherein the animal is a monkey.

34. The method of any one of claims 20-33, wherein the method further comprises determining the expression of a protein or mRNA of a gene in cells and/or tissue of the experimental animal.

35. The method of any one of claims 20-34, wherein the method further comprises determining cardiac metabolism, vascular metabolism, skeletal metabolism, kidney metabolism, liver metabolism, and or microbiome evaluation in the experimental animal.

36. The method of any one of claims 20-35, wherein the method further comprises administering a compound to the laboratory animal.

37. The method of claim 36, wherein the method further comprises determining the time course of the absorption of the compound, determining the biological distribution of the compound, determining the metabolism of the compound, and/or determining the excretion on the compound.

38. The method of any one of claims 20-37, wherein the method further comprises performing one or more assays selected from echocardiography, MRI, CT scan, SPECT/PET scan, NMR, left and right ventricles catheterization, hemodynamic studies, vascular stiffness measurement, blood glucose measurment, exercise testing, coronary flow reserve measurement, histological evaluation of cardiac morphology, skeletal muscle force measurement on the experimental animal or a biological sample from the experimental animal.
